(19) United States
(12) Patent Application Publication
(10) Pub. No.: US 2024/0398680 A1
(43) Pub. Date: Dec. 5, 2024

(54) ENHANCED DEPOSITION OF HAIR AND SKIN CONDITIONING INGREDIENTS USING HYDROXYPROPYL BISHYDROXYETHYLDIMONIUM CHLORIDE

(71) Applicant: COLONIAL CHEMICAL, INC., South Pittsburg, TN (US)

(72) Inventors: Robert N. Comber, South Pittsburg, TN (US); Dennis Abbeduto, South Pittsburg, TN (US); Jordan Taylor, South Pittsburg, TN (US); Molly McEnery, South Pittsburg, TN (US)

(21) Appl. No.: 18/683,236

(22) PCT Filed: Aug. 12, 2022
(86) PCT No.: PCT/US2022/040264

§ 371 Date: Feb. 12, 2024

Related U.S. Application Data
(60) Provisional application No. 63/232,579, filed on Aug. 12, 2021.

(51) Int. Cl. A61K 8/41; A61Q 5/02; A61Q 5/12; A61Q 19/10

(52) U.S. Cl. CPC A61K 8/416; A61Q 5/02; A61Q 5/12; A61Q 19/10; A61K 2800/5428; A61K 2800/596

(57) ABSTRACT

This invention relates to the use of Hydroxypropyl Bis-Hydroxyethyldimonium Chloride in hair and skin cleansing formulations that is useful in boosting the deposition of hair and skin conditioning agents.

### FIELD OF THE INVENTION

The present invention is directed to Hydroxypropyl Bis-Hydroxyethyldimonium Chloride compounds and formulations containing it that are surprising useful in boosting hair and skin conditioning. In one embodiment of the present invention, Hydroxypropyl Bis-Hydroxyethyldimonium Chloride is simply added to some existing market shampoos, body and facial washes to boost the deposition of conditioning agents and other beneficial materials.

### BACKGROUND OF THE INVENTION

Designing new, unique ingredients to boost the hair conditioning properties of shampoos and conditioners remains a target of the manufacturers of hair conditioning and cleansing products. As can be seen in the examples below, in one class of hair conditioning agents, they are designed with a long, aliphatic group attached to a quaternized nitrogen, or in some cases a tertiary amine that can be converted into its salt form, for example, a lactate salt.

U.S. Pat. No. 9,416,099, entitled “Quaternized Fatty Amines, Amidoamines and Their Derivatives From Natural Oil Metathesis, reports the design and synthesis of quaternary conditioning aids made from fatty sources derived from metatheses.

U.S. Pat. No. 10,617,619 relates to a composition comprising one or more fatty quaternary ester ammonium compounds and one or more fatty amidoamine compounds combined in a solid conditioner and a method to prepare the hair conditioner composition by dispersing the solid composition in water at moderate temperatures.

Other strategies have been used in the design of effective hair conditioning agents. Quaternized polysaccharide polymers or quaternized polysilicone polymers also provide hair conditioning effects from rinse off cleansing systems. A quaternized, high molecular weight silicone, for example, is disclosed in U.S. Pat. No. 8,263,061, and directed to a series of alkyl quaternium silicone compounds having both alkyl groups and fatty quaternary nitrogen groups attached in one molecule, and the intermediates formed during the production thereof, that are suitable for use in personal care and other applications.

Other strategies have been used in the design of cationic conditioning agents, but most strategies include the presence of a quaternary nitrogen along with a polymeric backbone or a long alkyl hydrophobe, the latter features promoting conditioning of the hair or skin surface.

Cola®Moist 200, or Hydroxypropyl Bis-Hydroxyethyldimonium Chloride (an ingredient for hair and skin care products manufactured by Colonial Chemical, Inc., South Pittsburgh, TN) is represented by the following structure:

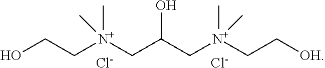 [SMILES: C[N+](C)(CCO)CC(O)C[N+](C)(C)CCO ; [Cl-] ; [Cl-]]

Cola®Moist 200 was designed as a cationic humectant with a high charge density and outstanding water binding properties. A few companies have found and patented other uses for Cola®Moist 200 based on its unique structure and properties.

For example, US Published Patent Application 2010/0158964, assigned to Kimberly-Clark, claims the use of Cola®Moist 200 as an emulsifying agent that helps create personal care compositions and personal care products that impart perceivable aesthetic benefits of increased softness, quietness and drapability to the skin or hair of a user.

3M Innovation Properties Company claims the use of Cola®Moist 200 in an adhesive masking article for shielding a protected work surface from a coating applied to a surface adjacent the protected work surface. See. For example, US Published Patent Applications 2013/0118402, 2010/0307411, and 2008/0300339.

Surprisingly, the present inventors have found that Cola®Moist 200, or Hydroxypropyl Bis-Hydroxyethyldimonium Chloride, a molecule that is not polymeric and that does not possess a long, conditioning alkyl group, promotes the deposition of hair and skin conditioning agents in cleansing formulations while, importantly, not possessing conditioning properties itself.

### SUMMARY OF THE INVENTION

As stated above, one embodiment of the present invention is a composition comprising a Hydroxypropyl Bis-Hydroxyethyldimonium Chloride compounds and formulations containing it that are surprisingly useful in boosting hair and skin conditioning.

One embodiment of the present invention is a coacervate composition that includes at least one cationic deposition polymer; a hydroxypropyl bis-hydroxyethyldimonium chloride coacervation enhancer; and an anionic surfactant.

In one aspect of the invention, the hydroxypropyl bis-hydroxyethyldimonium chloride coascervation enhancer is a compound of the following formula:

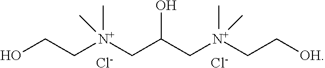 [SMILES: C[N+](C)(CCO)CC(O)C[N+](C)(C)CCO ; [Cl-] ; [Cl-]]

In another aspect, the cationic deposition polymer is selected from cationic guar gum derivatives, guar hydroxypropyltrimonium chloride, quaternary nitrogen-containing cellulose ethers, starch quats, silicone quats, polyquaternium-10, and combinations thereof.

In another aspect, the cationic disposition polymer is selected from the group consisting of cationic guars, cationic cellulose, cationic synthetic homopolymers, cationic synthetic copolymers, and combinations thereof. In yet another aspect, the cationic disposition polymer is selected from the group consisting of guar hydroxypropyltrimonium chloride, Polyquaternium 10, Polyquaternium 6, and combinations thereof.

In another aspect, the surfactant can include a primary surfactant and a co-surfactant. In embodiments of the invention, the surfactant may be sodium lauryl sulfate, sodium laureth-n sulfate, and combinations thereof.

Another embodiment of the invention is a coacervate composition that includes that includes at least one cationic deposition polymer; a hydroxypropyl bis-hydroxyethyldimonium chloride coascervation enhancer; an anionic surfactant; and at least one of a thickener, pearling agent, opacifier, colorant, preservative, pH adjuster, chelating agent.

Another embodiment of the invention is a method of enhancing or boosting the deposition of hair and skin conditioning agents, comprising providing a hair and skin conditioning agent; and adding a coacervate composition to the agent that comprises at least one cationic deposition polymer, a hydroxypropyl bis-hydroxyethyldimonium chloride coascervation enhancer, and an anionic surfactant to provide an enhanced hair and skin conditioning agent; and applying the enhanced hair and skin conditioning agent to the hair or skin.

In one aspect of this embodiment, the hair and skin conditioning agents are delivered from rinse off cleansing systems containing a high molecular weight conditioning agent. Also, the hair and skin conditioning agents are in a topical personal care composition. In aspects of the invention, the personal care composition is a shampoo, body washes, hair rinse or facial wash.

Also including in the present invention is a shampoo compositions that have at least one cationic deposition polymer; a hydroxypropyl bis-hydroxyethyldimonium chloride coascervation enhancer of the present invention; and an anionic surfactant.

### DESCRIPTION OF THE FIGURES

FIG. 1 is a graph that shows turbidity results for various cleansers containing increasing amounts of an enhancer of the present invention.

FIG. 2 is a graph that shows turbidity results comparing convention hair conditioning aids to an enhancer of the present invention for coacervate deposition.

FIG. 3 is a graph that shows deposition of Red 80 dye on wool treated with formulations in Table 4.

FIG. 4 is a graph that shows hair combing studies of hair tresses treated with and without an enhancer of the present invention.

### DESCRIPTION OF THE INVENTION

The details of one or more embodiments of the presently disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

While the terms used herein are believed to be well understood by those of ordinary skill in the art, certain definitions are set forth to facilitate explanation of the presently disclosed subject matter.

Unless defined otherwise, all technical and scientific terms used herein have the same meaning as is commonly understood by one of skill in the art to which the invention(s) belong.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which need to be independently confirmed.

The term “surfactant”, “surface active agent”, “surfactant”, or “dispersing agent” refers to an organic chemical that when added to a liquid changes the properties of that liquid at a surface.

The term “personal care composition” refers to compositions intended for topical application to skin or hair. Personal care compositions can optionally be rinse-off formulations, in which the product can be applied topically to the skin or hair and then subsequently rinsed within seconds to minutes from the skin or hair with water. The personal care compositions can be in the form of, for example, a liquid, semi-liquid cream, lotion, or gel and are intended for topical application to the skin and/or hair. Examples of personal care compositions can include but are not limited to bar soap, shampoo, conditioning shampoo, body wash, moisturizing body wash, shower gels, skin cleansers, cleansing milks, hair and body wash, in shower body moisturizer, pet shampoo, shaving preparations, and cleansing compositions used in conjunction with a disposable cleansing cloth.

Personal Care Compositions of the present invention can include a deposition polymer, an enhancer of the present invention, a surfactant and optional additional beneficial ingredients.

### Deposition Polymer

The personal care composition of the present invention can include a cationic polymer to allow formation of a coacervate. As can be appreciated, the cationic charge of a cationic polymer can interact with an anionic charge of a surfactant to form the coacervate.

A deposition polymer of the present invention may be one that is described in US Published Patent Application No. 2020/0188243. Accordingly, suitable cationic polymers can include: (a) a cationic guar polymer, (b) a cationic non-guar galactomannan polymer, (c) a cationic starch polymer, (d) a cationic copolymer of acrylamide monomers and cationic monomers, (e) a synthetic, non-crosslinked, cationic polymer, (f) cationic synthetic homopolymers, (g) a cationic cellulose polymer, and (h) combinations thereof. In certain examples, more than one cationic polymer can be included. The cationic polymer can be selected from guar hydroxypropyltrimonium chloride, Polyquaterium 10, Polyquaternium 6, and combinations thereof.

In certain embodiments, the deposition polymer may be a cationic polymer may be selected from the group consisting of cationic guars, cationic cellulose, cationic synthetic homopolymers, cationic synthetic copolymers, and combinations thereof.

In other embodiments, the deposition polymer may be selected from the group consisting of guar hydroxypropyltrimonium chloride, Polyquaternium 10, Polyquaternium 6, and combinations thereof.

A deposition polymer can be included by weight of the shampoo composition at about 0.05% to about 3%, about 0.075% to about 2.0%, or at about 0.1% to about 1.0%.

### Enhancer

The coacervate enhancer of the present invention is a Hydroxypropyl Bis-Hydroxyethyldimonium Chloride compound represented by the following structure:

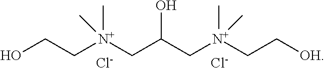 [SMILES: C[N+](C)(CCO)CC(O)C[N+](C)(C)CCO ; [Cl-] ; [Cl-]]

The enhancer of the present invention is known to be safe and non-irritating. It is non-greasy, water-soluble, and non-staining. It has also been found to be easily formulated into many emulsion products.

### Surfactant

The shampoo compositions described herein can include one or more surfactants in the surfactant system. The one or more surfactants can be selected from the group consisting of anionic surfactants, amphoteric surfactants, nonionic surfactants and mixtures thereof. The one or more surfactants can be substantially free of sulfate-based surfactants. As can be appreciated, surfactants provide a cleaning benefit to soiled articles such as hair, skin, and hair follicles by facilitating the removal of oil and other soils. Surfactants generally facilitate such cleaning due to their amphiphilic nature which allows for the surfactants to break up, and form micelles around, oil and other soils which can then be rinsed out, thereby removing them from the soiled article. Suitable surfactants for a shampoo composition can include anionic moieties to allow for the formation of a coacervate with a cationic polymer. The surfactant may be chosen from the group consisting of anionic surfactants, amphoteric surfactants, nonionic surfactants, and mixtures thereof.

Suitable anionic detersive surfactants include those which are known for use in hair care or other personal care shampoo compositions. The anionic detersive surfactant may be a combination of sodium lauryl sulfate and sodium laureth-n sulfate. The concentration of the anionic surfactant in the composition should be sufficient to provide the desired cleaning and lather performance, and generally range from about 5% to about 50%, alternatively from about 8% to about 30%, alternatively from about 9% to about 25%, and alternatively from about 10% to about 17%, by weight of the composition. The anionic surfactant may be selected from the group consisting of sodium, ammonium or potassium salts of isethionates; sodium, ammonium or potassium salts of alkyl sulfates; sodium, ammonium or potassium salts of alkyl ethoxylate sulfates; sodium, ammonium or potassium salts of sulfonates; sodium, ammonium or potassium salts of ether sulfonates; sodium, ammonium or potassium salts of sulfosuccinates; sodium, ammonium or potassium salts of sulfoacetates; sodium, ammonium or potassium salts of glycinates; sodium, ammonium or potassium salts of sarcosinates; sodium, ammonium or potassium salts of glutamates; sodium, ammonium or potassium salts of alaninates; sodium, ammonium or potassium salts of carboxylates; sodium, ammonium or potassium salts of taurates; sodium, ammonium or potassium salts of phosphate esters; and combinations thereof.

In other embodiments of the present invention, examples of additional anionic surfactants suitable for use include, but are not limited to, ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, monoethanolamine cocoyl sulfate, sodium trideceth sulfate, sodium tridecyl sulfate, sodium methyl lauroyl taurate, sodium methyl cocoyl taurate, sodium lauroyl isethionate, sodium cocoyl isethionate, sodium laurethsulfosuccinate, sodium laurylsulfosuccinate, sodium tridecyl benzene sulfonate, sodium dodecyl benzene sulfonate, and mixtures thereof.

### Co-Surfactant

Personal care compositions of the present invention can optionally include a co-surfactant. Co-surfactants are materials which are combined with the anionic surfactants to enhance lather volume and/or to modify lather texture or reduce irritation. Typically these materials can be selected from a variety of families of structures including, but not limited to, amphoteric, zwitterionic, cationic, and nonionic. They are typically used with anionic surfactants in a weight ratio of 1:20 to 1:4, and alternatively in the 1:12 to 1:7 weight ratio.

The shampoo composition may comprise from about 0.5 wt % to about 10 wt %, alternatively from about 0.5 wt % to about 5 wt %, alternatively from about 0.5 wt % to about 3 wt %, alternatively from about 0.5 wt % to about 2 wt %, and alternatively from about 0.5 wt % to about 1.75 wt % by weight of the composition of at least one suitable co-surfactant. The co-surfactant may serve to produce faster lather, facilitate easier rinsing, and/or mitigate harshness on the keratinous tissue. The co-surfactant further may aid in producing lather having more desirable texture, volume and/or other properties.

### Liquid Carrier

The personal care compositions of the present invention can also include a liquid carrier. Inclusion of an appropriate quantity of a liquid carrier can facilitate the formation of a shampoo composition having an appropriate viscosity and rheology. A shampoo composition can include, by weight of the composition, about 60% to about 95% of a liquid carrier, about 65% to about 92%, about 70% to about 90% of a liquid carrier, and about 75% to about 90% of a liquid carrier.

A liquid carrier can be water or can be a miscible mixture of water and organic solvent. A liquid carrier can be water with minimal or no significant concentrations of organic solvent, except as otherwise incidentally incorporated into the composition as minor ingredients of other essential or optional components. Suitable organic solvents can include water solutions of lower alkyl alcohols and polyhydric alcohols. Useful lower alkyl alcohols include monohydric alcohols having 1 to 6 carbons, such as ethanol and isopropanol. Exemplary polyhydric alcohols include propylene glycol, hexylene glycol, glycerin, and propane diol.

### Other Optional Ingredients

As can be appreciated, the personal care compositions of the present invention described herein can include a variety of optional components to tailor the properties and characteristics of the composition. As can be appreciated, suitable optional components are well known and can generally include any components which are physically and chemically compatible with the essential components of the shampoo compositions described herein. Optional components should not otherwise unduly impair product stability, aesthetics, or performance. Individual concentrations of optional components can generally range from about 0.001% to about 10%, by weight of a shampoo composition.

Suitable optional components which can be included in a shampoo composition can include natural ingredients such as, tea extracts, and natural antioxidants such as grape seed extracts, natural hair conditioning oils, such as safflower oil, jojoba oil, argan oil, and combinations thereof.

Suitable optional components which can be included in a shampoo composition can include co-surfactants, deposition aids, cationic polymers, conditioning agents (including hydrocarbon oils, fatty esters, silicones), anti-dandruff agents, suspending agents, viscosity modifiers, dyes, nonvolatile solvents or diluents (water soluble and insoluble), pearlescent aids, foam boosters, pediculocides, pH adjusting agents, perfumes, preservatives, chelants, proteins, skin active agents, sunscreens, UV absorbers, and vitamins.

Additional other ingredients can also be added to the personal care composition for treatment of the skin, or to modify the aesthetics of the personal care composition as is the case with perfumes, colorants, dyes or the like, that are well known in the art.

Additional other ingredients can also be beneficial materials including fragrances, silicones, solid particles, and other hydrophobic skin/hair conditioning ingredients.

Other non-limiting ingredients that can be used in the personal care compositions include components that can be selected from the group consisting of thickening agents; preservatives; antimicrobials; fragrances; chelators (e.g., such as those described in U.S. Pat. No. 5,487,884 issued to Bisset, et al.); sequestrants; vitamins (e.g., Retinol); vitamin derivatives (e.g., tocophenyl actetate, niacinamide, panthenol); sunscreens; desquamation actives (e.g., such as those described in U.S. Pat. Nos. 5,681,852 and 5,652,228 issued to Bisset); anti-wrinkle/anti-atrophy actives (e.g., N-acetyl derivatives, thiols, hydroxyl acids, phenol); anti-oxidants (e.g., ascorbic acid derivatives, tocophenol) skin soothing agents/skin healing agents (e.g., panthenoic acid derivatives, aloe vera, allantoin); skin lightening agents (e.g., kojic acid, arbutin, ascorbic acid derivatives) skin tanning agents (e.g., dihydroxyacteone); anti-acne medicaments; essential oils; sensates; pigments; colorants; pearlescent agents; interference pigments (e.g., such as those disclosed in U.S. Pat. No. 6,395,691 issued to Liang Sheng Tsaur, U.S. Pat. No. 6,645,511 issued to Aronson, et al., U.S. Pat. No. 6,759,376 issued to Zhang, et al, U.S. Pat. No. 6,780,826 issued to Zhang, et al.) particles (e.g., talc, kolin, mica, smectite clay, cellulose powder, polysiloxane, silicas, carbonates, titanium dioxide, polyethylene beads) hydrophobically modified non-platelet particles (e.g., hydrophobically modified titanium dioxide and other materials described in a commonly owned, patent application published on Aug. 17, 2006 under Publication No. 2006/0182699A, entitled “Personal Care Compositions Containing Hydrophobically Modified Non-platelet particle filed on Feb. 15, 2005 by Taylor, et al.) and mixtures thereof. The multiphase personal care composition can comprise from about 0.1% to about 4%, by weight of the personal care composition, of hydrophobically modified titanium dioxide. Other such suitable examples of such skin actives are described in U.S. Patent Application Publication No. 2012/0009285. Other ingredients can be most typically those materials approved for use in cosmetics and that are described in the CTFA Cosmetic Ingredient Handbook, Second Edition, The Cosmetic, Toiletries, and Fragrance Association, Inc. 1988, 1992.

As can be appreciated, a shampoo composition can include still further optional components. For example, amino acids can be included. Suitable amino acids can include water soluble vitamins such as vitamins B1, B2, B6, B12, C, pantothenic acid, pantothenyl ethyl ether, panthenol, biotin, and their derivatives, water soluble amino acids such as asparagine, alanin, indole, glutamic acid and their salts, water insoluble vitamins such as vitamin A, D, E, and their derivatives, water insoluble amino acids such as tyrosine, tryptamine, and their salts.

A shampoo composition can include pigment materials such as inorganic, nitroso, monoazo, disazo, carotenoid, triphenyl methane, triaryl methane, xanthene, quinoline, oxazine, azine, anthraquinone, indigoid, thionindigoid, quinacridone, phthalocianine, botanical, natural colors, including: water soluble components such as those having C. I. Names. The compositions can also include antimicrobial agents which are useful as cosmetic biocides and antidandruff agents including: water soluble components such as piroctone olamine, water insoluble components such as 3,4,4′-trichlorocarbanilide (trichlosan), triclocarban and zinc pyrithione.

One or more stabilizers and preservatives can be included. For example, one or more of trihydroxystearin, ethylene glycol distearate, citric acid, sodium citrate dihydrate, a preservative such as kathon, sodium chloride, sodium benzoate, and ethylenediaminetetraacetic acid (“EDTA”) can be included to improve the lifespan of a shampoo composition.

### Experimental Results

The unexpected impact of Cola®Moist 200 on the deposition of hair conditioning agents was demonstrated using three methods:
1) Turbidity (haziness) measurements were taken over a range of dilutions at ambient temperature to show the impact that Cola®Moist 200 has on coacervate formation in Polyquaternium-10 based formulations.
2) Rubine dye deposition was measured colorimetrically on wool swatches to demonstrate the substantivity of conditioning agents in the presence of Cola®Moist 200.
3) Finally, hair combing studies were used to measure combing force reduction on wet, lightly bleached brown hair.

### Turbidity Measurements

Coacervation refers to the generation and separation of an insoluble complex within a cleansing solution of anionics and cationics [see George Deckner, “Understanding Conditioning from Rinse Off Products, Prospector Knowledge Center; Deposition from Rinse Off Products|Prospector (ulprospector.com)]. “Coacervation occurs when anionic surfactant/cationic polymer complexes that are soluble in the product precipitate during product dilution or rinsing

Coacervate formation is dependent upon a variety of criteria such as molecular weight, concentration, and ratio of interacting ionic materials, ionic strength (including modification of ionic strength, for example, by addition of salts), charge density of the cationic and anionic species, pH, and temperature. Coacervate systems and the effect of these parameters have been described by J. Caelles, et al., “Anionic and Cationic Compounds in Mixed Systems”, Cosmetics & Toiletries, Vol. 106, April 1991, pp 49-54, C. J. van Oss, “Coacervation, Complex-Coacervation and Flocculation”, J Dispersion Science and Technology, Vol. 9 (5,6), 1988-89, pp 561-573, and D. J. Burgess, “Practical Analysis of Complex Coacervate Systems”, J of Colloid and Interface Science, Vol. 140, No. 1, November 1990, pp 227-238].

Coacervate formation is indicated by haze and therefore a dilution-deposition mechanism is desired, in which the cleanser is clear and coacervate complexes are generated by the dilution during the washing and rinsing processes while cleansing, resulting in conditioning effects. To determine the potential for coacervate formation during cleansing, turbidity measurements were taken on the cleanser over a range of dilutions at ambient temperature.

The base formula, or control formula, for the turbidity measurements is seen below in Table 1.

TABLE 1
Cleanser formula for Turbidity Measurements

| Ingredient | Control |
|---|---|
| Water | 35.70 |
| Polymer JR30 | 0.50 |
| (Polyquaternium-10) | |
| ColaTeric 2C | 6.80 |
| (Disodium | |
| Cocoamphodiacetate) | |
| Colonial SLES-2 | 57.00 |
| (Sodium Laureth Sulfate) | |
| Cola ®Moist 200 | 0-2% Active |
| Citric Acid, 50% | qs to pH 6 |

[Cola ®Moist 200 is sold as a 70% actives solution in water. It was diluted to 30% to be in line with the concentrations of the rest of the test substances].

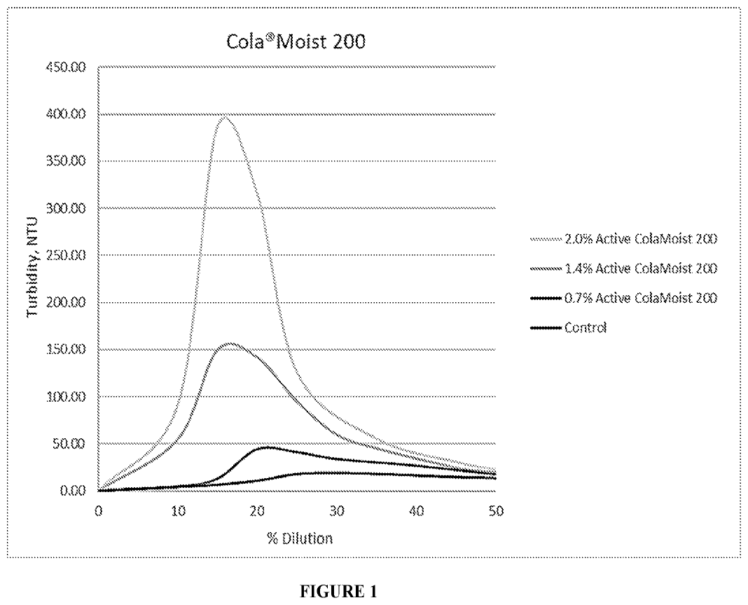

Cleansing samples were placed in a polished sample cell and analyzed with an automatic turbidimeter [A Hach 2100P Turbidimeter; Hach Company, 5600 Lindbergh Dr, Loveland, CO 80538]. Samples were compared to the control sample containing no Cola®Moist 200. The results of these studies are tabulated in Table 2 and shown in FIG. 1.

TABLE 2
Turbidity results for various shampoos containing
increasing amounts of Cola ®Moist 200.
Turbidity Results

Cola ®Moist 200

| % Dilution | Control | 1% As Is | 2% As Is | 2% Active |
|---|---|---|---|---|
| 0 | 0.00 | 0.00 | 0.00 | 0.00 |
| 10 | 3.99 | 4.81 | 54.50 | 91.30 |
| 15 | 6.48 | 12.80 | 150.00 | 387.00 |
| 20 | 10.60 | 44.10 | 142.00 | 316.00 |
| 25 | 17.80 | 40.90 | 95.00 | 126.00 |
| 30 | 18.90 | 33.70 | 59.80 | |
| 35 | 17.90 | | 45.20 | 55.40 |
| 40 | | 26.60 | | |
| 45 | 14.60 | | 24.60 | 30.60 |
| 50 | 13.50 | 17.30 | 19.60 | 22.40 |

Increasing amounts of Cola®Moist 200 added to a cleansing formulation result in increased coacervate formation as depicted by the line graphs in FIG. 1. As alluded to above, this is typically seen with materials like hydrophobically modified cationic materials like quaternary ammonium hair conditioning agents. This is generally not the case for simple diquats like Cola®Moist 200 that do not contain a hydrophobic group.

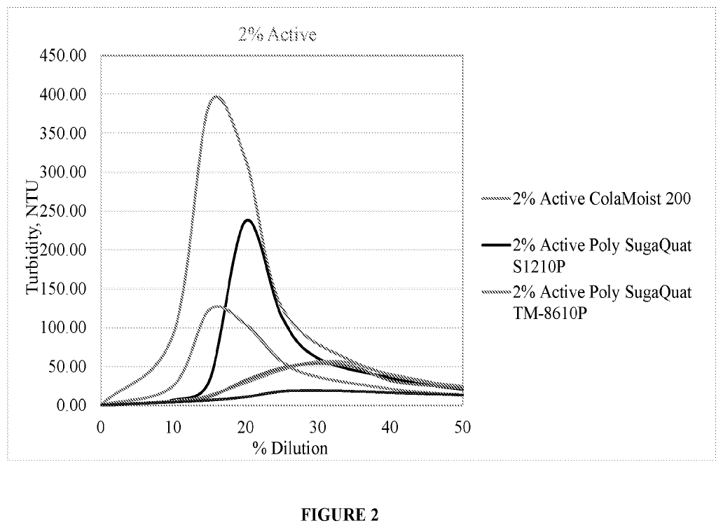

Table 3 and FIG. 2 shows how Cola®Moist 200 compares to other materials in the ability to elicit this type of coacervate phenomenon. Table 3 is the cleansing formula that various quaternary materials were added to. As is seen in FIG. 2, Cola®Moist 200 outperforms other conventional hair conditioning aids in promoting the generation of a coacervate. All these conventional hair conditioning aids contain a hydrophobic region in their chemical make-up. Cola®Moist 200 has none.

TABLE 3
General cleansing formula that various cationic
conditioning aids were added to (2% actives)

| Ingredient | Control |
|---|---|
| Water | 29.03 |
| Polymer JR30M | 0.50 |
| (Polyquaternium-10) | |
| ColaTeric 2C | 6.80 |
| (Disodium | |
| Cocoamphodiacetate) | |
| Colonial SLES-2 | 57.00 |
| (Sodium Laureth Sulfate) | |
| Test products in FIG. 2 | 6.68 (2% Active) |
| (30% solutions) | |
| Citric Acid, 50% | qs to pH 6 |

The other conditioning aids shown in FIG. 2 are the following: Poly Suga®Quat S1210P, or Polyquaternium-81 (30% actives), sold by Colonial Chemical, Inc.; Poly Suga®Quat TM-8610P, or Polyquaternium-77 (30% actives), sold by Colonial Chemical, Inc.; Cola®Lipid SAFL (30% actives), or Linoleamidopropyl PG-dimonium chloride phosphate, sold by Colonial Chemical, Inc.; and Cola®Lipid GS (30% actives), or Grapeseed Amidopropyl PG-dimonium chloride phosphate, sold by Colonial Chemical, Inc. Again, all contain a hydrophobic portion in the make-up of their structure].

### Red 80 Dye Test

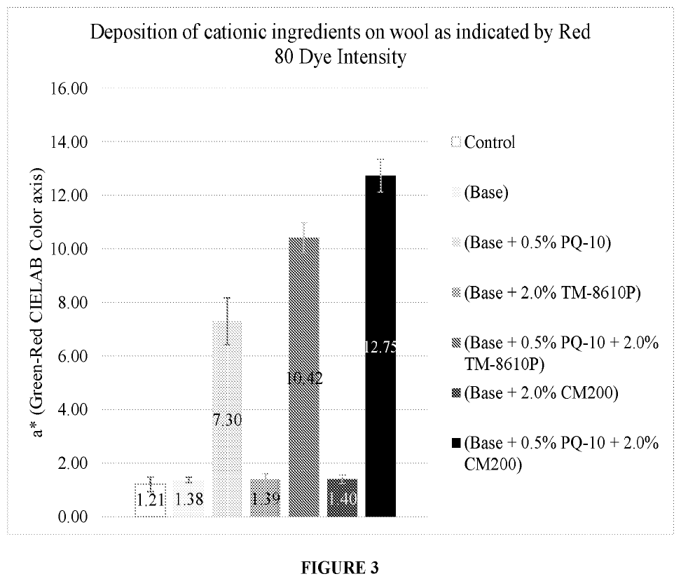

The Red 80 dye test predicts the deposition of cationic substances on keratin by measuring the amount of an anionic red dye that gets deposited on a wool swatch in a wash event compared to the same wool material washed without the cationic polymer [R. J. Crawford, C. R. Robbins, J. Soc. Cosmet. Chem., 31, 273-278 (September/October 1980)]. For each of the products tested, a 2″×2″ wool swatch is washed under a standard wash procedure and then color differences are determined using the green-red axis color value, a*, of CIE L*a*b* coordinates measured with a Hunter Labs MiniScan 4000S. Increased a* values denote increased red intensity, thereby indicating greater amount of red dye deposition and therefore increased cationic deposition. This would be expected if the amount of coacervate, as measured by the turbidity experiment, was a good indicator of substantivity. The formulations that were used to treat wool swatches are shown in Table 4. The results of our dye deposition study are depicted in FIG. 3.

TABLE 4
Formulations used to treat wool swatches and demonstrate cationic substantivity

| | | | | | 2% | 2% |
|---|---|---|---|---|---|---|
| | | | | | Actives | Actives |
| | | Formula | 2% | 2% | Formula + | Formula + |
| | Base | with 0.5% | Actives | Actives | 0.5% | 0.5% |
| Ingredient | Formula | PQ-10 | Formula | Formula | PQ-10 | PQ-10 |
| Water | 52.15 | 51.65 | 45.48 | 45.48 | 44.98 | 44.98 |
| Cola ® Moist 200 | — | — | 6.67 | | 6.67 | — |
| (30%) | | | | | | |
| Poly Suga ® Quat | — | — | | 6.67 | — | 6.67 |
| TM-8610P (30%) | | | | | | |
| Polymer JR30M | | 0.5 | | | 0.5 | 0.5 |
| (Polyquaternium-10) | | | | | | |
| Colonial SLES-2 | 42.75 | 42.75 | 42.75 | 42.75 | 42.75 | 42.75 |
| (Sodium Laureth | | | | | | |
| Ether Sulfate) | | | | | | |
| Cola ® Teric 2C | 5.10 | 5.10 | 5.10 | 5.10 | 5.10 | 5.10 |
| (Disodium | | | | | | |
| Cocoamphodiacetate) | | | | | | |
| Citric Acid (50%) | q.s. to | q.s. to | q.s. to | q.s. to | q.s. to | q.s. to |
| | pH 6 | pH 6 | pH 6 | pH 6 | pH 6 | pH 6 |

Poly Suga®Quat TM-8610P, or Polyquaternium-77, is a patented hair and skin conditioning ingredient manufactured by Colonial Chemical, Inc., South Pittsburgh, TN, and is sold at a 30% solids concentration. Cola®Moist 200, or Hydroxypropyl Bis-Hydroxyethyldimonium Chloride (a hair and skin care ingredient manufactured by Colonial Chemical, Inc., South Pittsburgh, TN) is sold as a 70% solids product, but was diluted to 30% solids for the Red Dye 80 study. In the formulations evaluated 6.67% of the two above cationics (each 30% solids solutions) were added, providing 2% cationic solids overall for promoting deposition.

In FIG. 3, the Control formula is water which would not be expected to exhibit any Red Dye 80 deposition. The Base Formula listed in Table 4 with no cationics would also not be expected to exhibit dye deposition on wool. The first formula to exhibit any substantivity to wool was the base formula containing Polyquaternium-10 (PQ-10). Compare to the other cationic ingredients evaluated, it had the best performance because of its polymeric nature. Note that when Poly Suga®Quat TM-8610P (Polyquaternium-77) or Cola®Moist 200 (Hydroxypropyl Bis-Hydroxyethyldimonium Chloride were added to the formulation without the presence of PQ-10, there was essentially no Red Dye 80 deposited. In the absence of coacervate formation, these materials do not deposit on wool. They do not exhibit deposition on their own. However, when Poly Suga®Quat TM-8610P (Polyquaternium-77) was added to the formula in addition to PQ-10, a noticeable increase in Red Dye 80 deposition was measured, in line with the amount of coacervate noted earlier. Similarly, when Cola®Moist 200 (Hydroxypropyl Bis-Hydroxyethyldimonium Chloride) was added to the base formula with PQ-10, it also gave a noticeable, and slightly more intense Red Dye 80 deposition than Poly SugaRQuat TM-8610P as measured by Hunter Labscan Colorimeter. These results are also in line with the Turbidity measurements reported above in which the addition of Cola®Moist 200 resulted in increased turbidity, an indicator of enhanced coacervate formation and the potential for more deposition (and perhaps an increase in potential for combing or skin feel benefits). The hair combing studies below will confirm the benefits that enhanced deposition brings to hair conditioning.

It is important to note that the mechanism by which Cola®Moist 200 is exerting its effect on hair conditioning is not by being a conditioning agent, but by promoting the deposition of conditioning agents, namely the coacervate formed by PQ-10, the anionic surfactants and any other materials that a coacervate may bring to the surface of hair or skin.

### Hair Combing Studies

Enhanced deposition of Red Dye 80 on wool, as well as enhanced coacervate formation in rinse shampoo systems, were leading indicators that Cola®Moist 200 might boost the hair conditioning from cleansing rinse off formulations. To confirm the conditioning boosting benefits of Cola®Moist 200, some of the above formulations were tested using a fibre.one tress testing instrument from Dia-Stron (Andover, Hampshire, United Kingdom). The fibre.one tress testing instrument is an instrument that accurately measures the amount of tension created when hair is pulled through a mounted conventional comb. It can also be used to measure hold, stiffness, feel, smoothness or surface damage.

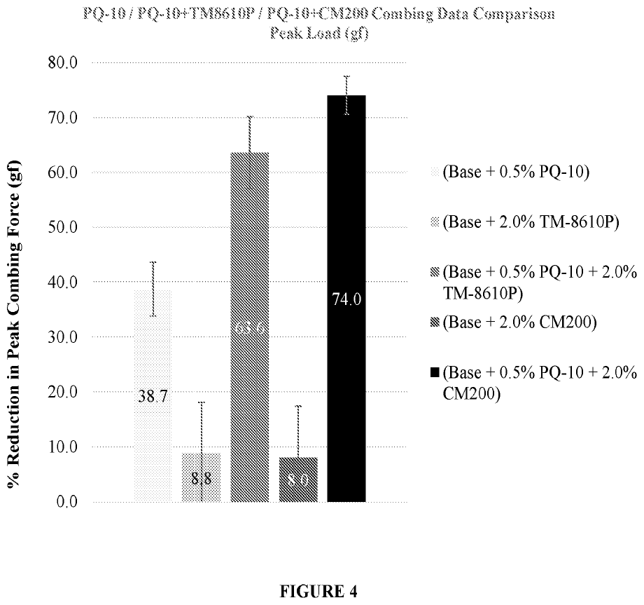

In our studies, regular bleached hair tresses purchased from International Hair Importers, Glendale, NY were used. Each tress was 2 g of hair, 6″ long and 1″ wide, bound on one end with hot melt. The results for the formulations measured are shown in FIG. 4. In the evaluations 0.4 g of each formula was applied to a wet tress and after 1 minute of working/lathering the shampoo into the tress, they were rinsed using in-house established rinse protocols and mounted in the fibre.one combing apparatus. The tresses were combed 11 times according to established protocols and 10 of 11 data points were averaged for the reduction in peak combing force seen in FIG. 4.

Inspection of FIG. 4 first shows that neither Polyquaternium-77 nor Cola®Moist 200 on their own provide any meaningful combing benefit. FIG. 4 does show that the base shampoo formula with only PQ-10 added resulted in a combing force reduction of 38.7% compared to the base shampoo formula alone. When 2% active of Polyquaternium-77 was added to the base formula already containing PQ-10, this resulted in a 63.6% reduction of combing force. When Cola®Moist 200 was added to the base formula already containing PQ-10, this resulted in a 74% reduction in combing force. These results are in line with the Red Dye 80 deposition experiments above as well as the coacervate formation experiments above. Again, on its own, Cola®Moist 200 does not show much deposition on keratin and does not act alone as a conditioning agent.

Based on its simple structure below of a diquat possessing no hydrophobic group, it is unexpected that the enhancer of the present invention would show hair combing benefit or any participation in coacervate formation in rinse off systems that would lead to dilution deposition on hair and reduced combing forces in hair combing studies. However, the combing results confirm that Cola®Moist 200 provides an unexpected hair conditioning boosting benefit from cleansing systems. To date, Cola®Moist 200 has been positioned as a cationic humectant, a skin and hair moisturizing ingredient, outperforming glycerin in its ability to moisturize. We now report and confirm the unexpected results of Cola®Moist 200 as an ingredient that can enhance the deposition of coacervates and hair conditioning agents onto hair and skin from cleansing systems with measurable conditioning benefits. Although Colonial has taught the use of Cola®Moist 200 as a humectant and moisturizing aid and example formulations appear on its website, no formulations have been published or taught that contain Cola®Moist 200 in the presence of Polyquaternium-10 for participation in coacervate formation. It is highly likely that Cola®Moist 200 will also promote coacervate formation, and hence the deposition of conditioning aids, in the presence of other higher molecular weight cationic polymers such as guar quats, starch quats, and silicone quats to name some that are commonly used in personal care products.

The invention thus being described, it will be apparent to those skilled in the art that various modifications and variations can be made in the present invention without departing from the scope or spirit of the invention. Other aspects of the invention will be apparent to those skilled in the art from consideration of the specification and practice of the invention disclosed herein. It is intended that the specification and examples be considered as exemplary only.

We claim:

1. A coacervate composition comprising:
at least one cationic deposition polymer; and
a hydroxypropyl bis-hydroxyethyldimonium chloride coacervation enhancer; and
an anionic surfactant.

2. The coacervate composition of claim 1, wherein the hydroxypropyl bis-hydroxyethyldimonium chloride coascervation enhancer is a compound of the following formula:

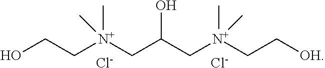 [SMILES: C[N+](C)(CCO)CC(O)C[N+](C)(C)CCO ; [Cl-] ; [Cl-]]

3. The composition of claim 1, wherein the cationic deposition polymer is selected from cationic guar gum derivatives, guar hydroxypropyltrimonium chloride, quaternary nitrogen-containing cellulose ethers, starch quats, silicone quats, polyquaternium-10, and combinations thereof.

4. The composition of claim 1, wherein the cationic disposition polymer is selected from the group consisting of cationic guars, cationic cellulose, cationic synthetic homopolymers, cationic synthetic copolymers, and combinations thereof.

5. The composition of claim 1, wherein the cationic disposition polymer is selected from the group consisting of guar hydroxypropyltrimonium chloride, Polyquaternium 10, Polyquaternium 6, and combinations thereof.

6. The composition of claim 1, wherein the cationic deposition polymer is polyquaternium-10.

7. The composition of claim 1, wherein the at least one surfactant includes a primary surfactant and a co-surfactant.

8. The composition of claim 1, wherein the surfactant is chosen from sodium lauryl sulfate, sodium laureth-n sulfate, and combinations thereof.

9. A cleansing formulation comprising a coacervate composition of claim 1 and at least one of a thickener, pearling agent, opacifier, colorant, preservative, pH adjuster, chelating agent.

10. A method of enhancing or boosting the deposition of hair and skin conditioning agents, comprising:
providing a hair and skin conditioning agent;
adding a coacervate composition to the agent that comprises at least one cationic deposition polymer, a hydroxypropyl bis-hydroxyethyldimonium chloride coascervation enhancer, and an anionic surfactant to provide an enhanced hair and skin conditioning agent; and
applying the enhanced hair and skin conditioning agent to the hair or skin.

11. The method of claim 10, wherein the hair and skin conditioning agents are delivered from rinse off cleansing systems containing a high molecular weight conditioning agent.

12. The method of claim 10, wherein the hair and skin conditioning agents are in a topical personal care composition.

13. The method of claim 10, wherein the personal care composition is a shampoo, body washes, hair rinse or facial wash.

14. The method of claim 10, wherein the hydroxypropyl bis-hydroxyethyldimonium chloride coacervation enhancer is a compound of the following formula:

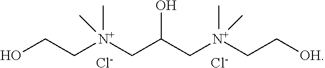 [SMILES: C[N+](C)(CCO)CC(O)C[N+](C)(C)CCO ; [Cl-] ; [Cl-]]

15. The method of claim 10, wherein the cationic deposition polymer is selected from cationic guar gum derivatives, guar hydroxypropyltrimonium chloride, quaternary nitrogen-containing cellulose ethers, starch quats, silicone quats, polyquaternium-10, and combinations thereof.

16. The method of claim 10, wherein the cationic deposition polymer is polyquaternium-10.

17. The method of claim 10, wherein the composition comprises at least one of a primary surfactant, a co-surfactant, thickener, pearling agent, opacifier, colorant, preservative, pH adjuster, chelating agent.

18. A shampoo composition, comprising:
at least one cationic deposition polymer; and
a hydroxypropyl bis-hydroxyethyldimonium chloride coacervation enhancer; and
an anionic surfactant.

19. The shampoo composition of claim 18, further comprising a carrier.

20. The shampoo composition of claim 18, wherein the hydroxypropyl bis-hydroxyethyldimonium chloride coacervation enhancer is a compound of the following formula: 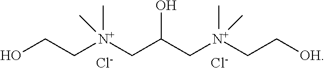 [SMILES: C[N+](C)(CCO)CC(O)C[N+](C)(C)CCO ; [Cl-] ; [Cl-]]
